(19) United States
(12) Patent Application Publication
(10) Pub. No.: US 2007/0244503 A1
(43) Pub. Date: Oct. 18, 2007

(54) METHOD FOR FILTERING EMBOLIC MATERIAL

(75) Inventors: Brendan Casey, Barna (IE); John Neilan, Gort (IE); Gary Fahey, Rosscahill (IE); Michael Gilvarry, Ballina (IE); Eamon Brady, Elphin (IE); David Vale, Dublin (IE); Steven Horan, Athlone (IE); Ronald Kelly, Cannistown (IE); Gerard McCaffrey, Galway (IE)

Correspondence Address: SUGHRUE MION, PLLC, 2100 PENNSYLVANIA AVENUE, N.W., SUITE 800, WASHINGTON, DC, 20037, US

(73) Assignee: SALVIAC LIMITED

(21) Appl. No.: 11/279,759

(22) Filed: Apr. 14, 2006

(51) Int. Cl. A61M 29/00

(52) U.S. Cl. 606/200

(57) ABSTRACT

In a method for filtering embolic material, a guide catheter (5) is advanced through a vasculature. A delivery catheter (4), a guidewire (2) and a collapsed filter are advanced together through the guide catheter (5) to a point distally of the distal end of the guide catheter (5). The guidewire (2) is then advanced across the lesion. During this advancement of the guidewire (2), the delivery catheter (4) and the collapsed filter remain substantially stationary. When the guidewire (2) has crossed the lesion, the delivery catheter (4) and the collapsed filter are then advanced across the lesion until the collapsed filter is distal of the lesion. The delivery catheter (4) is then withdrawn to facilitate deployment of the filter at the location distal of the lesion to ensure that any embolic material released during performance of an interventional procedure at the lesion will be captured and safely retained within the filter.

### INTRODUCTION

This invention relates to a method for filtering embolic material for blood flowing in a vasculature during an interventional procedure.

It is known to perform an interventional procedure, such as an angioplasty balloon dilation or placement of a stent, at a region of stenosis in a vasculature. Performing such an interventional procedure may result in embolic material being released from the region of stenosis and entering the bloodstream. If this embolic material were permitted to travel downstream, the embolic material may result in potentially fatal consequences.

This invention is aimed at providing a method which addresses this problem.

### STATEMENTS OF INVENTION

According to the invention there is provided a method for filtering embolic material from blood flowing in a vasculature during an interventional procedure, the method comprising the steps of:
providing an embolic protection filter having at least one proximal inlet and a plurality of distal outlets which are sized to capture embolic material while allowing blood to flow, the filter having a collapsed delivery configuration and an expanded configuration;
providing a guidewire, the filter being moveable relative to the guidewire;
advancing the filter in the collapsed delivery configuration through a vasculature to an intermediate location proximal of a desired treatment location;
advancing the guidewire through the vasculature from the intermediate location;
crossing the desired treatment location with the guidewire;
advancing the filter in the collapsed delivery configuration over the guidewire; and
deploying the filter distal to the treatment location.

In one embodiment of the invention the method comprises:

providing a delivery catheter for containing the filter in the collapsed configuration; and

advancing the delivery catheter, the filter and the guidewire to the intermediate location. The method may comprise providing a guide catheter at an entry into a vasculature; and advancing the filter and guidewire through the guide catheter to the intermediate location. The intermediate location may be adjacent to a distal end of the guide catheter. The intermediate location may be distal to the distal end of the guide catheter. The intermediate location may be proximal to the distal end of the guide catheter. The intermediate location may be between the distal end of the guide catheter and the desired treatment location.

In one case the method comprises the steps of:
advancing the guidewire from a first intermediate location to a further intermediate location which is proximal of the treatment location and, subsequently,
advancing the filter in the collapsed delivery configuration over the guidewire toward the further intermediate location.

The method may comprise repeating these steps for additional intermediate locations.

In one case the treatment location is in the carotid artery. The treatment location may be at or adjacent to the carotid bifurcation. An intermediate location may be at or adjacent to the aortic arch. An intermediate location may be at or adjacent to the carotid take-off

In another embodiment the method comprises:
providing the filter in the expanded configuration; and
loading the filter into the delivery catheter so that the delivery catheter contains the filter in the collapsed configuration.

In one case the method comprises providing a guidewire extending through the filter in the delivery catheter. The filter and the delivery catheter may be advanced along the guidewire. The method may further comprise the step of removing the delivery catheter from the filter at the deployment location. Stored energy in the filter may expand the filter on removal of the delivery catheter from the filter. The method may comprise withdrawing the delivery catheter from the deployment location. The delivery catheter may be withdrawn from the deployment location after the deployment of the filter.

In another case the method comprises introducing an interventional catheter over the guidewire to the treatment location for carrying out an interventional procedure, embolic material generated during the treatment procedure being captured by the deployed filter.

The treatment location may be a region of stenosis. The interventional procedure may include a balloon dilation of the stenosis while the filter is deployed. The interventional procedure may include placing a stent at the treatment location while the filter is deployed.

In one case the method further comprises the steps of:
removing the interventional catheter from the treatment location; and
advancing a capture sheath over the guidewire.

The method may further comprise the steps of:
engaging the filter with the capture sheath; and
withdrawing the filter and the capture sheath from the treatment location.

In one embodiment the method further comprises the step of withdrawing the filter from the treatment location. The method may further include the step of withdrawing the guidewire after withdrawal of the filter.

The filter may be slidably disposed on the guidewire when the filter is in an expanded deployed configuration. The filter may be rotatably disposed on the guidewire when the filter is in an expanded deployed configuration. The filter may be mounted to a tubular member. The tubular member may comprise a collar. The tubular member may extend distally of the filter. At least one stop may be disposed on the guidewire. The at least one stop may be a distal stop disposed on the guidewire distally of the filter.

In another embodiment the filter comprises a filter body and a filter support which supports the filter body in the deployed configuration. The filter may expand from energy stored in the collapsed filter. The filter may expand from energy stored in a filter support made from a memory material. The memory material may be an alloy. The alloy may be Nitinol. The filter support may comprise at least one loop.

In one case the method comprises moving the filter relative to the delivery catheter to load the filter into the delivery catheter. The method may comprise pushing the filter into the delivery catheter. The method may comprise pulling the filter into the delivery catheter. The method may comprise the step of flushing the filter and/or the delivery catheter. The filter and/or delivery catheter may be flushed prior to loading of the filter into the delivery catheter. The filter and/or the delivery catheter may be flushed during loading of the filter into the delivery catheter. The filter and/or the delivery catheter may be flushed after loading of the filter into the delivery catheter.

In one case the step of moving the embolic protection filter relative to the catheter causes an automatic flushing of the embolic protection filter and/or the delivery catheter. The filter may be immersed in a flushing liquid before loading. The method may comprise the step of sealing the embolic protection filter immersed in the flushing liquid. The step of moving the embolic protection filter relative to the catheter may cause at least some of the flushing liquid to move relative to the embolic protection filter and/or relative to the catheter.

### BRIEF DESCRIPTION OF THE DRAWINGS

The invention will be more clearly understood from the following description of some embodiments thereof, given by way of example only, with reference to the accompanying drawings, in which:

FIGS. 1 to 9 are views illustrating a method for filtering embolic material according to one embodiment of the invention;

FIGS. 10 to 15 are views illustrating a method for filtering embolic material according to another embodiment of the invention;

FIGS. 16 to 21 are views illustrating a method for filtering embolic material according to another embodiment of the invention; and

FIGS. 22 to 31 are views illustrating a method for filtering embolic material according to a further embodiment of the invention.

### DETAILED DESCRIPTION

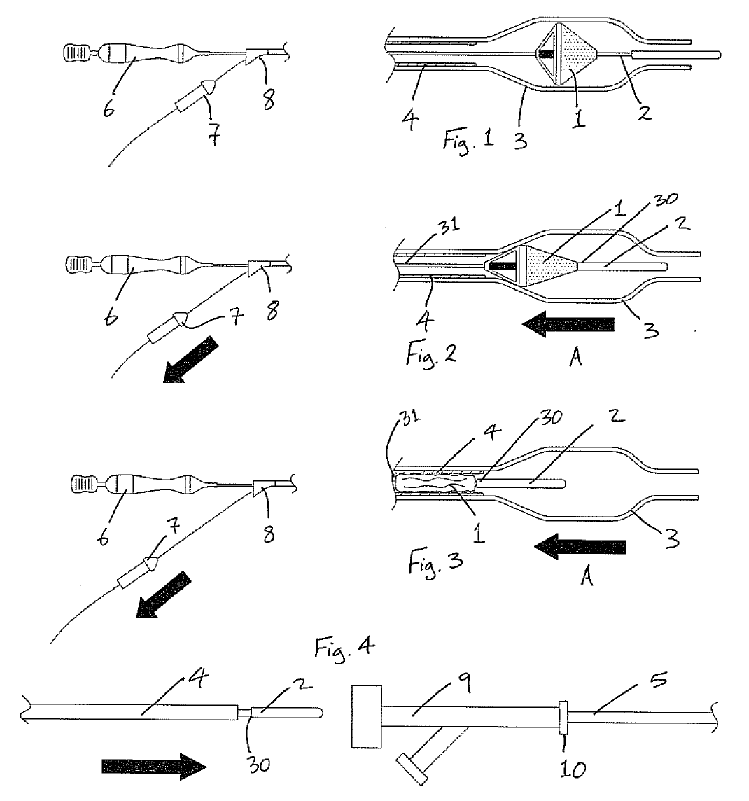

Referring to FIGS. 1 to 9 there is illustrated an embolic protection system being employed in a method for filtering embolic material according to the invention. The embolic protection system in accordance with the present invention includes a collapsible filter member 1 disposed upon a guidewire 2, wherein the collapsible filter member is configured to be disposed within a delivery/retrieval catheter 4.

In accordance with the present invention and as shown in FIG. 1, an embolic protection filter 1 is disposed upon a guidewire 2. The distal end of the guidewire 2 includes a feature 30 formed thereon, wherein the feature 30 is configured to retain the embolic protection filter 1 thereon. The feature 30 is preferably formed as an enlarged diameter member, wherein the diameter of the feature 30 is greater than an aperture of the embolic protection filter 1 through which the guidewire 2 is disposed.

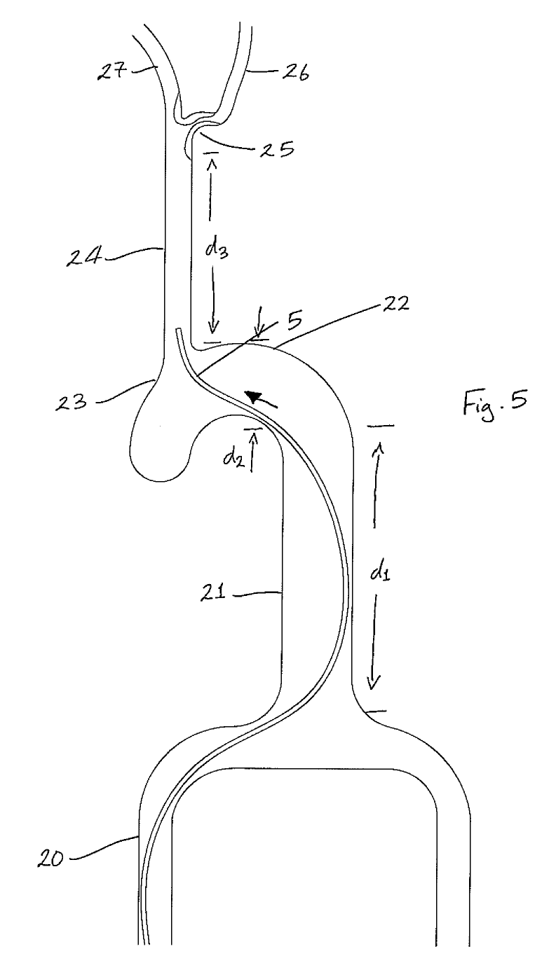

As shown in FIG. 1, the guidewire 2 and the embolic protection filter 1 are disposed within a flushing housing 3, the flushing housing 3 having a proximal end and a distal end and a space defined therebetween, wherein the space defined between the proximal and distal ends is sized to receive the embolic protection filter 1 in an uncompressed state. The filter 1 is loaded into a delivery catheter 4 by pulling the guidewire 2 proximally as shown in FIGS. 2 and 3, wherein the proximal end of the flushing housing 3 is shaped to facilitate the collapsing of the embolic protection filter 1. For example, as shown, the flushing housing 3 may be shaped to include a tapered portion, wherein as the embolic protection filter 1 and the guidewire 2 are drawn proximally, the tapered shape of the flushing housing 3 causes the embolic protection filter 1 to collapse to a diameter sufficiently small enough wherein the embolic protection filter 1 and the guidewire 2 can then be drawn into a delivery catheter 4. The delivery catheter 4, the guidewire 2 and the collapsed filter 1 are ready for insertion into a guide catheter 5 (FIG. 4).

Examples of suitable filters for use with the methods in accordance with the present invention are described in co-pending patent applications having Ser. Nos. 10/442,115 and/or 11/141,709 and/or 10/325,954 the entireties of which are herein incorporated by reference.

In further detail, FIG. 1 illustrates the delivery catheter handle 6, and a torque device 7. Wherein the handle 6 is associated with the proximal end of the delivery catheter 4, and the torque device 7 is associated with the proximal end of the guidewire 2.

FIG. 2 illustrates the loading/flushing funnel housing 3 as described above, wherein the embolic protection filter 1 and the guidewire 2 are shown disposed within the housing 3. As shown by arrow A, the embolic protection filter 1 and the guidewire 2 are moved proximally relative to the housing 3, wherein, the embolic protection filter 1 is collapsed by the tapered walls of the housing 3 as shown.

FIG. 3 illustrates a tapered hoop port 8, wherein the tapered hoop port 8 is disposed adjacent the handle 6. The tapered hoop port 8 is in communication with a lumen 31 of the delivery catheter 4, wherein as shown in FIG. 3, the proximal end of the guidewire 2 is disposed through the tapered hoop port 8.

FIG. 4 illustrates the delivery catheter 4, the filter delivery wire 2, a Touhy borst 9, the guide catheter 5. As shown in FIG. 4, the Touhy borst 9 is associated with the proximal end of the guide catheter 5 and in fluid communication with a lumen of the guide catheter 5, the lumen of the guide catheter 5 being sized to receive the delivery catheter 4 of the present invention.

FIG. 5 illustrates the femoral arteries 20, the aorta 21, the aortic arch 22, the left carotid take-off 23, the common carotid 24, the carotid bifurcation 25, the internal carotid 26, the external carotid 27. The distance d1 is approximately 50 cm, the distance d2 is approximately 25 mm, the distance d3 is approximately 15 cm. It shall be understood that the distances described herein are merely exemplary and will vary according to different anatomies.

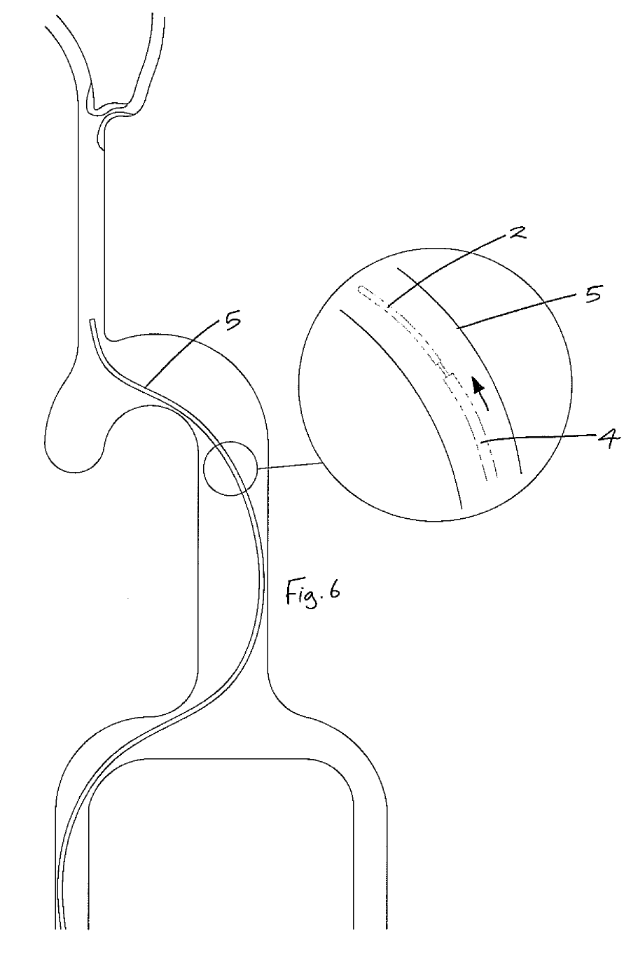

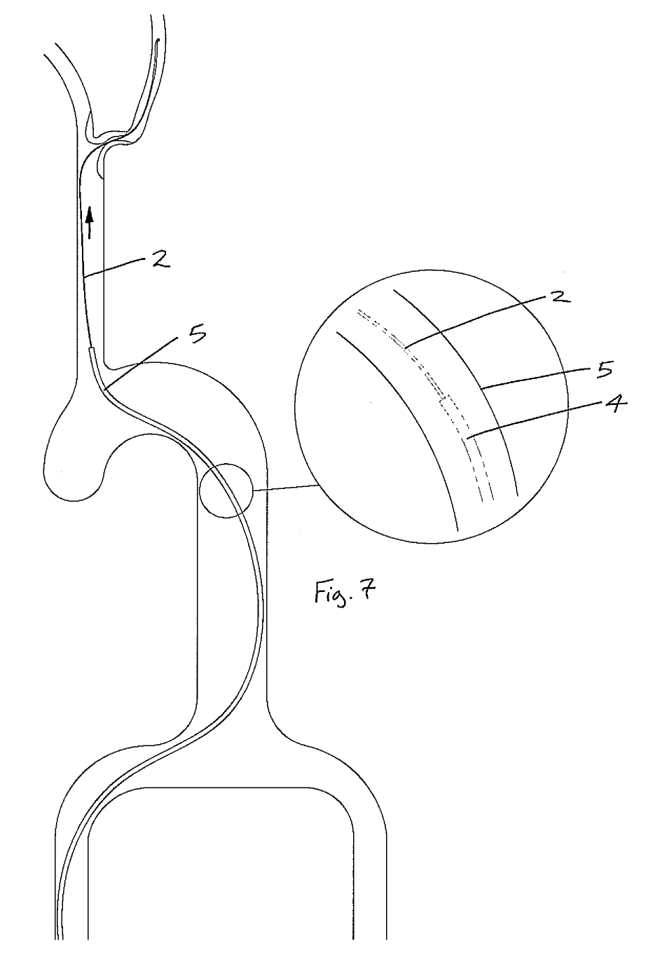

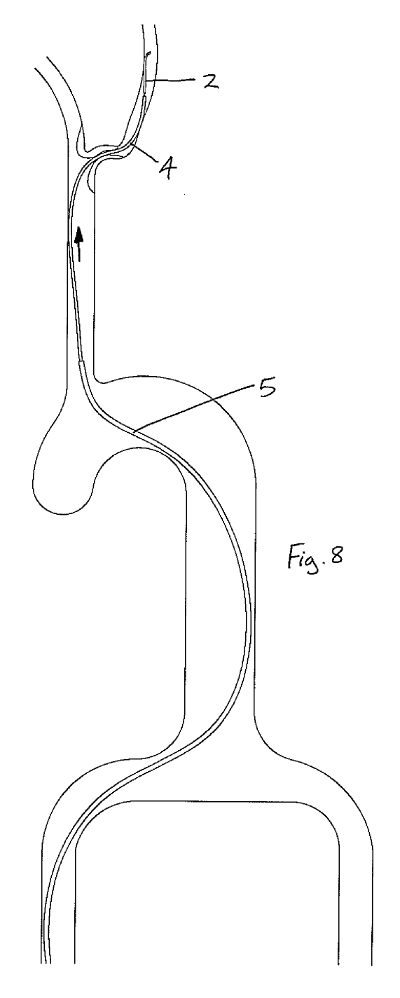

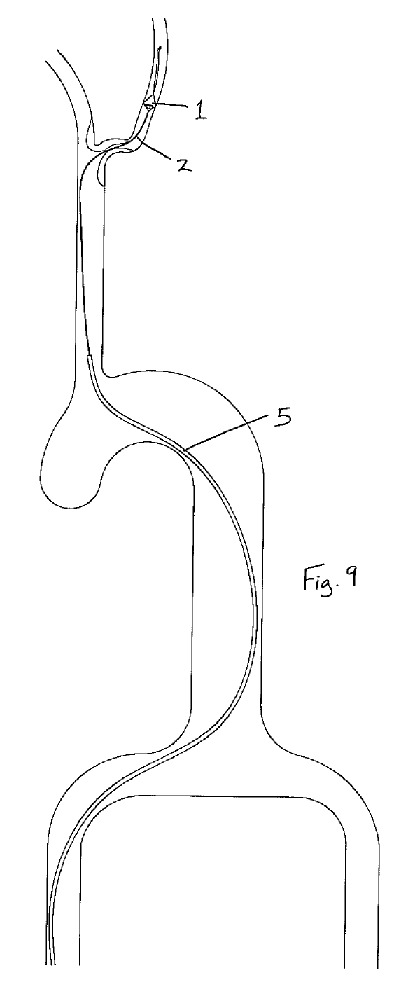

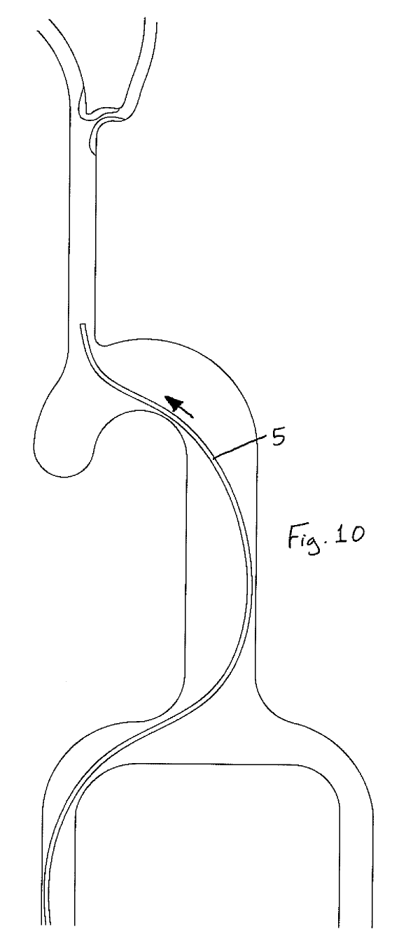

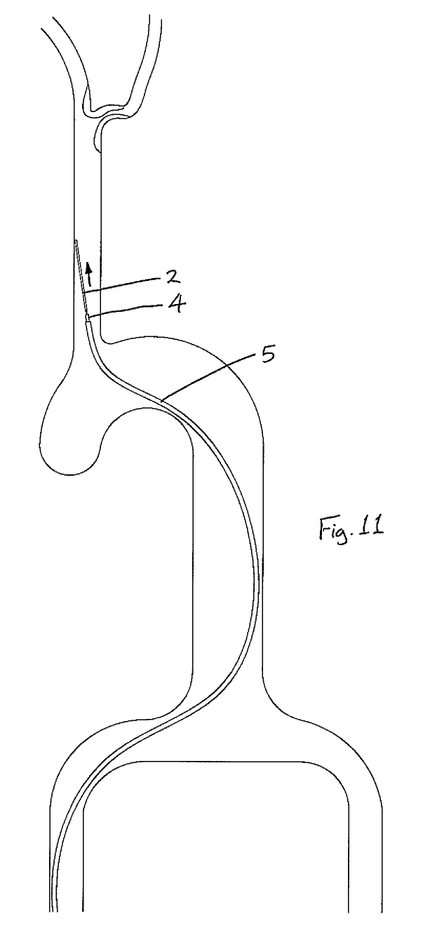

The guide catheter 5 is advanced through a vasculature (FIG. 5). The delivery catheter 4, the guidewire 2 and the collapsed filter 1 are advanced together through the guide catheter 5 to a point proximally of the distal end of the guide catheter 5 (FIG. 6). The guidewire 2 is then advanced out of the guide catheter 5 across the lesion (FIG. 7). During this advancement of the guidewire 2, the delivery catheter 4 and the collapsed filter 1 remain within the guide catheter 5. When the guidewire 2 has crossed the lesion, the delivery catheter 4 and the collapsed filter 1 are then advanced out of the guide catheter 5 and across the lesion until the collapsed filter 1 is distal of the lesion (FIG. 8). The delivery catheter 4 is then withdrawn relative to the collapsed filter 1 to facilitate deployment of the filter 1 at the location distal of the lesion to ensure that any embolic material released during an interventional procedure at the lesion will be captured and be safely retained within the filter 1 (FIG. 9). The filter 1 may be deployed between about 1 mm and about 100 mm distal the lesion and preferably the filter 1 is deployed approximately about 15 mm distal of the lesion at the carotid bifurcation 25.

In FIGS. 10 to 15 there is illustrated the embolic protection system being employed in an alternative method for filtering embolic material according to the invention.

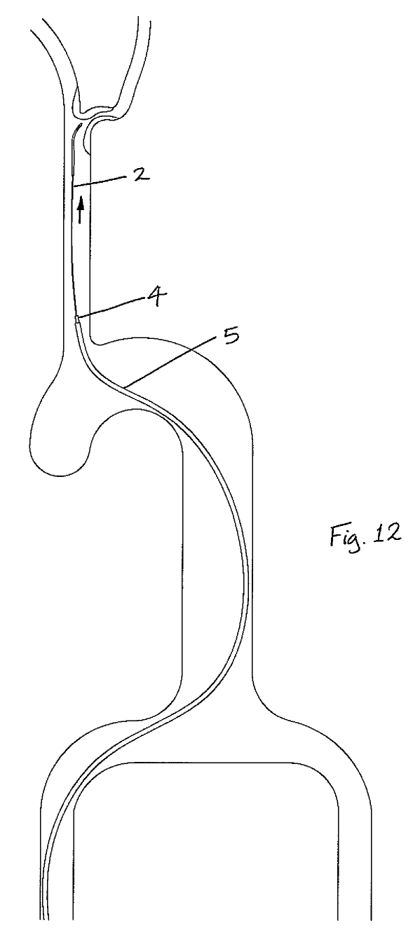

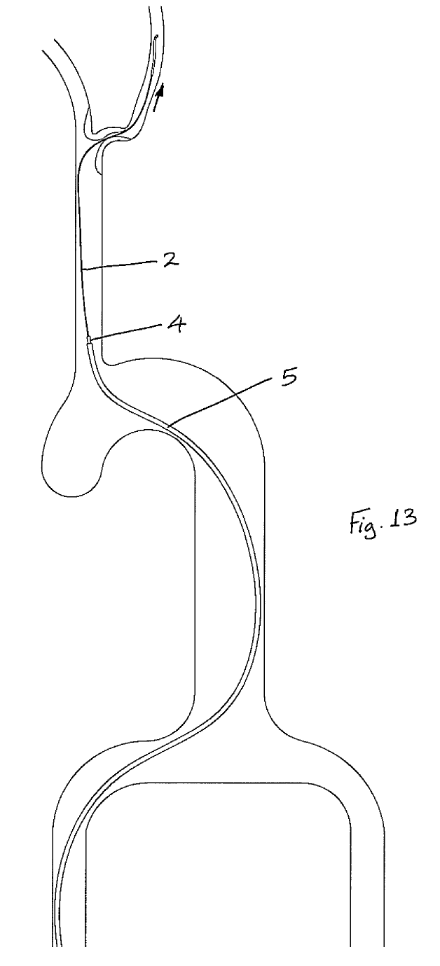

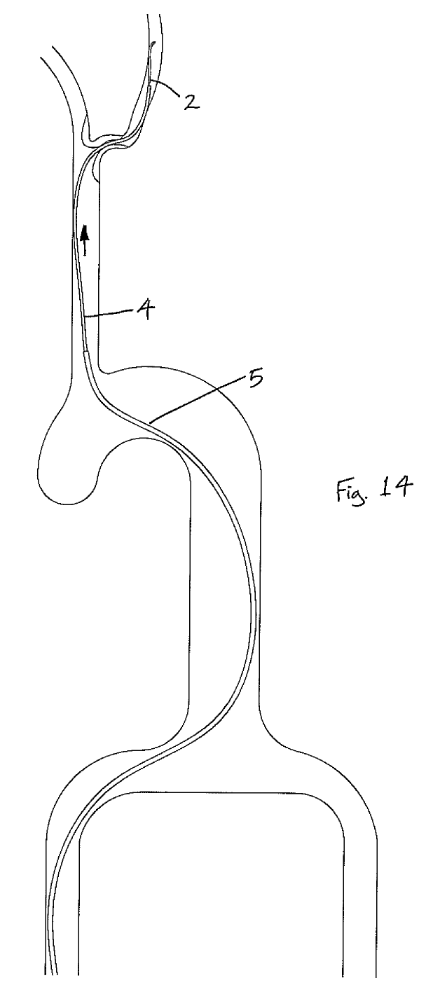

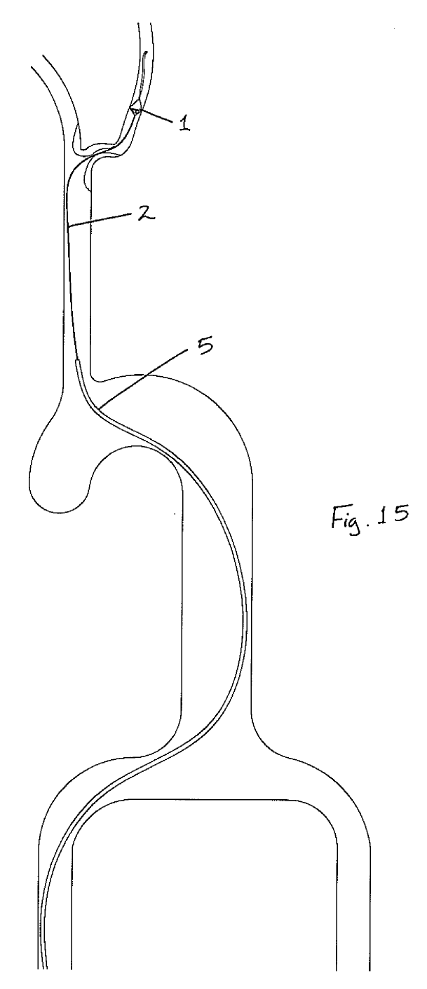

In this case, the guide catheter 5 is advanced through a vasculature (FIG. 10). The delivery catheter 4, the guidewire 2 and the collapsed filter 1 are advanced together through the guide catheter 5 to a point distally of the distal end of the guide catheter 5 (FIG. 11). The guidewire is then advanced across the lesion (FIGS. 12 and 13). During advancement of the guidewire 2, the delivery catheter 4 and the collapsed filter 1 remain substantially stationary. When the guidewire 2 has crossed the lesion, the delivery catheter 4 and the collapsed filter 1 are then advanced across the lesion until the collapsed filter 1 is distal of the lesion (FIG. 14). The delivery catheter 4 is then withdrawn relative to the collapsed filter 1 to facilitate deployment of the filter 1 at the location distal of the lesion to ensure that any embolic material released during performance of an interventional procedure at the lesion will be captured and safely retained within the filter 1 (FIG. 15).

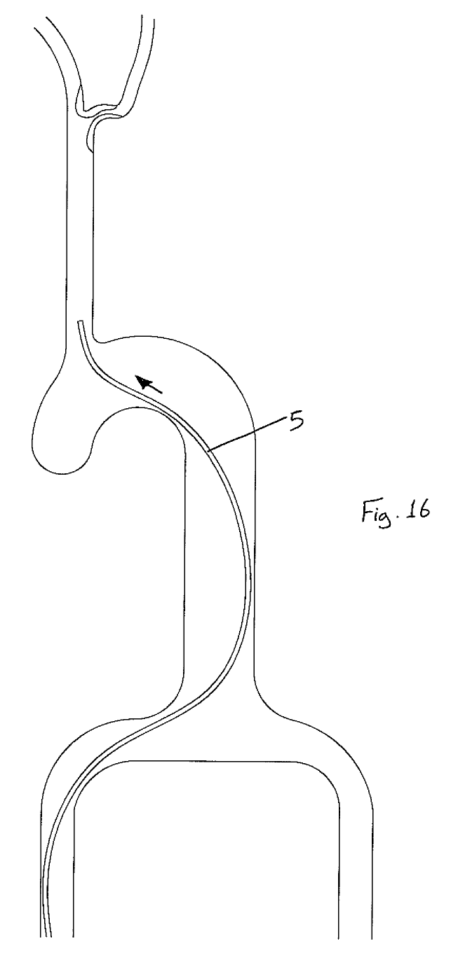

FIGS. 16 to 21 illustrate the embolic protection system being employed in another alternative method for filtering embolic material according to the invention.

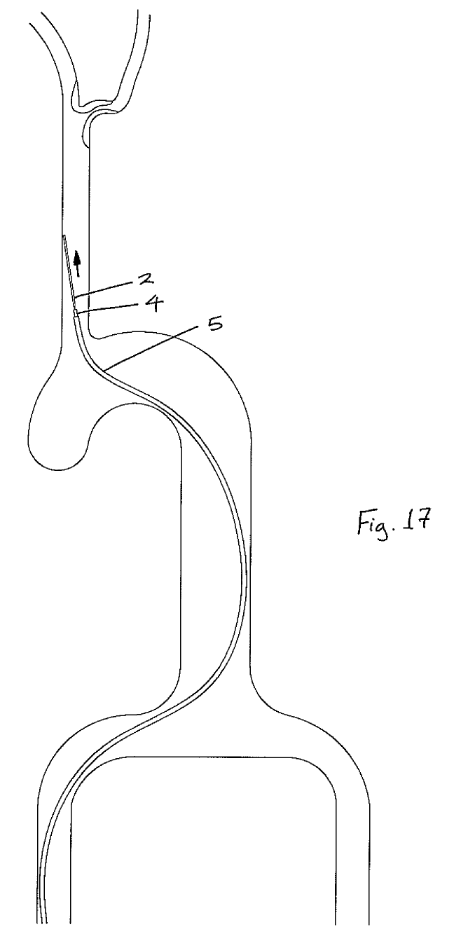

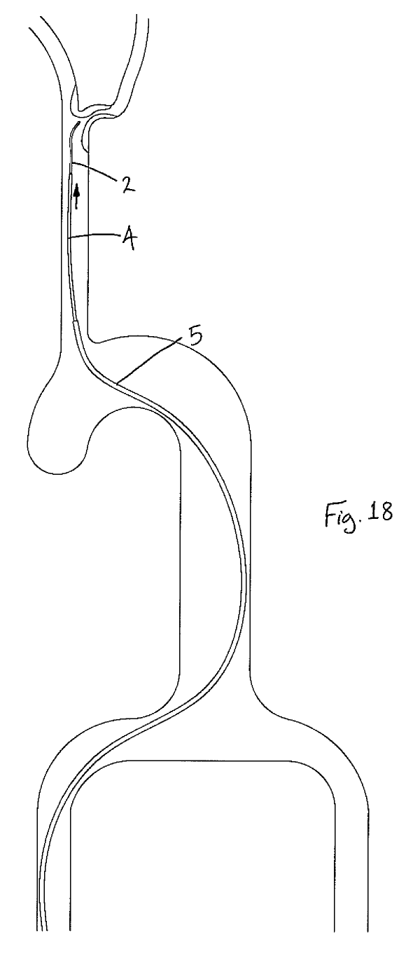

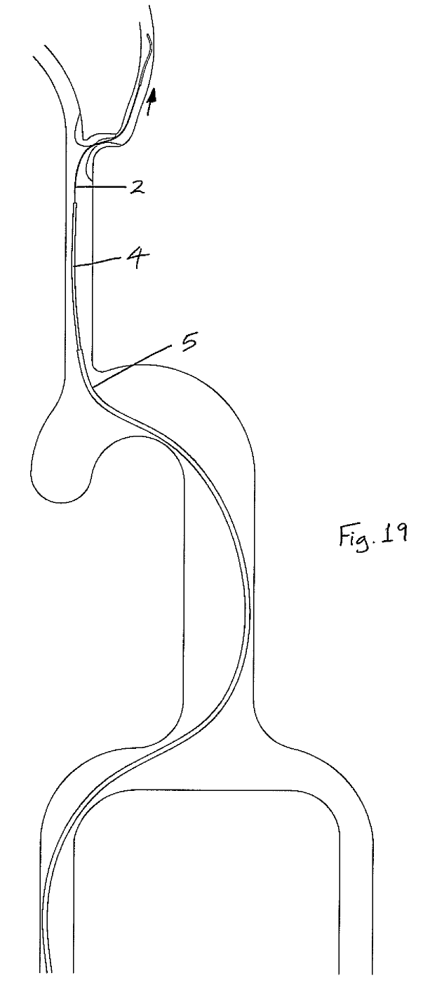

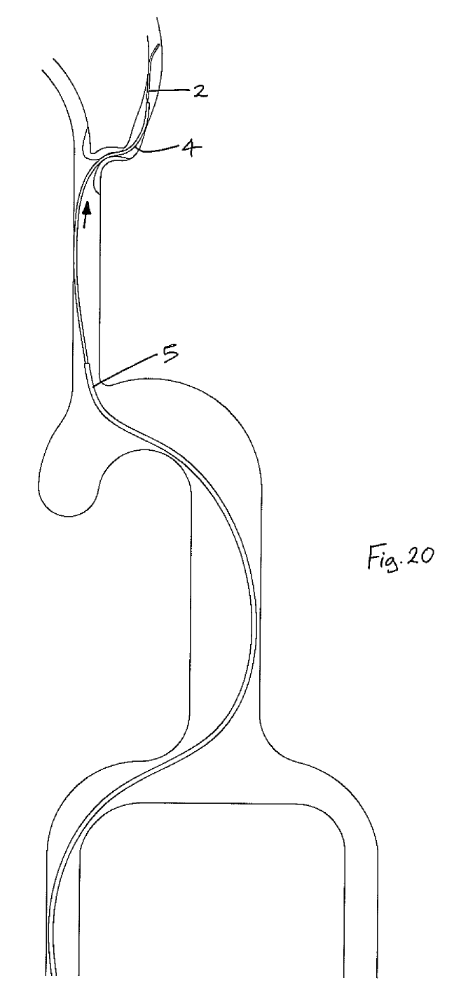

In this case the guide catheter 5 is advanced through a vasculature (FIG. 16). The delivery catheter 4, the guidewire 2 and the collapsed filter 1 are advanced together through the guide catheter 5 to a point distally of the distal end of the guide catheter 5 (FIG. 17). The delivery catheter 4, the guidewire 2 and the collapsed filter 1 are then advanced together further distally to a point immediately proximally of the lesion (FIG. 18). The guidewire 2 is then advanced across the lesion (FIG. 19). During this advancement of the guidewire 2, the delivery catheter 4 and the collapsed filter 1 remain substantially stationary. When the guidewire 2 has crossed the lesion, the delivery catheter 4 and the collapsed filter 1 are then advanced across the lesion until the collapsed filter 1 is distal of the lesion (FIG. 20). The delivery catheter 4 is then withdrawn relative to the embolic protection filter 1 to facilitate deployment of the filter 1 at the location distal of the lesion to ensure that any embolic material released during performance of an interventional procedure at the lesion will be captured and safely retained within the filter 1 (FIG. 21).

Referring to FIGS. 22 to 31 there is illustrated the embolic protection system being employed in a further alternative method for filtering embolic material according to the invention.

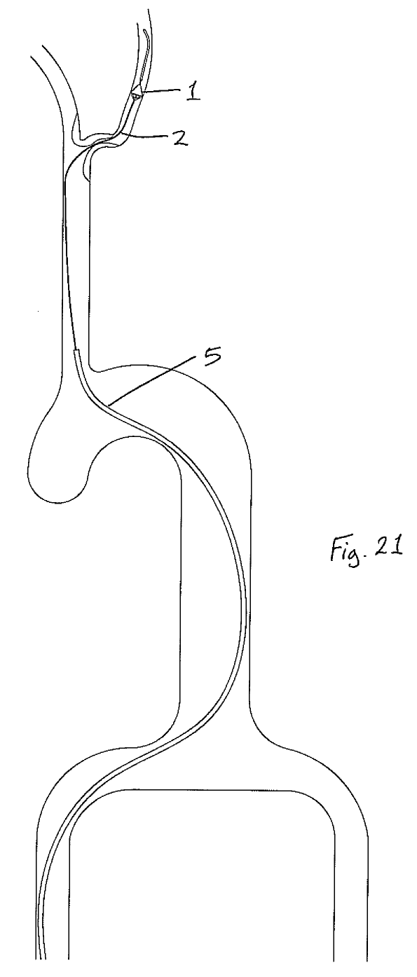

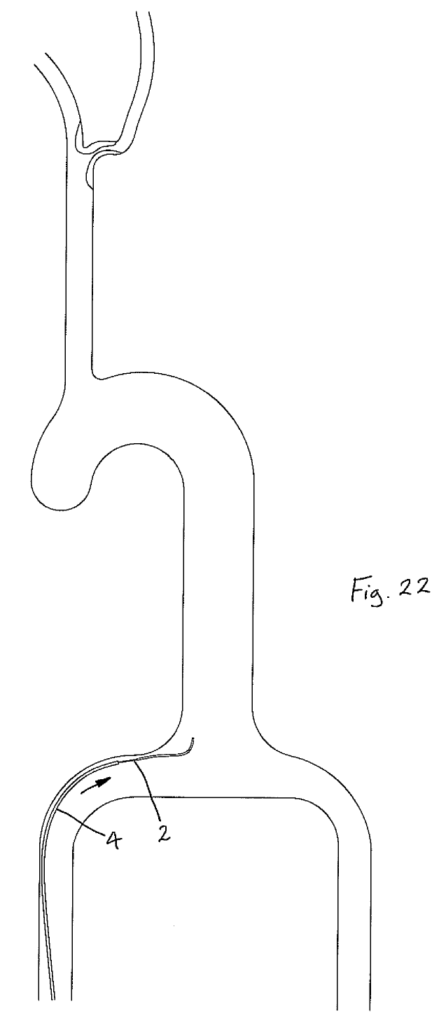

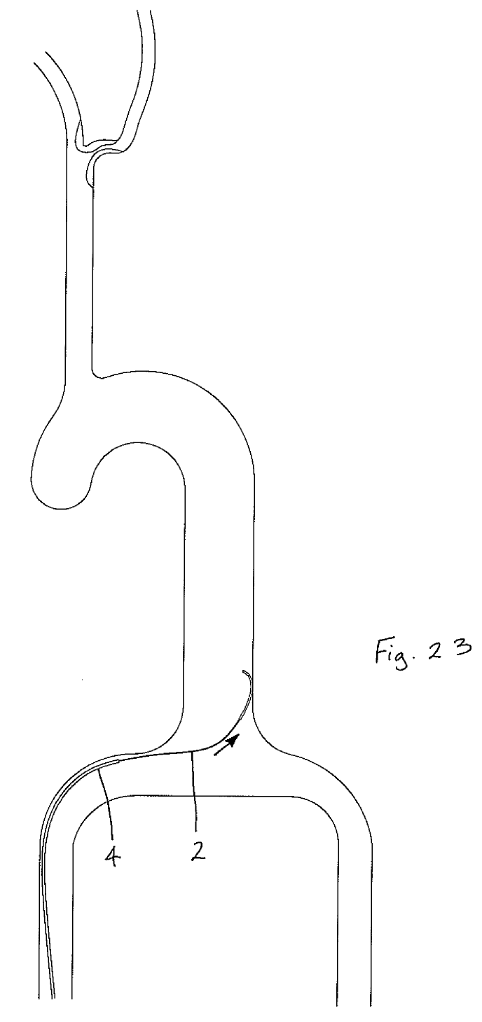

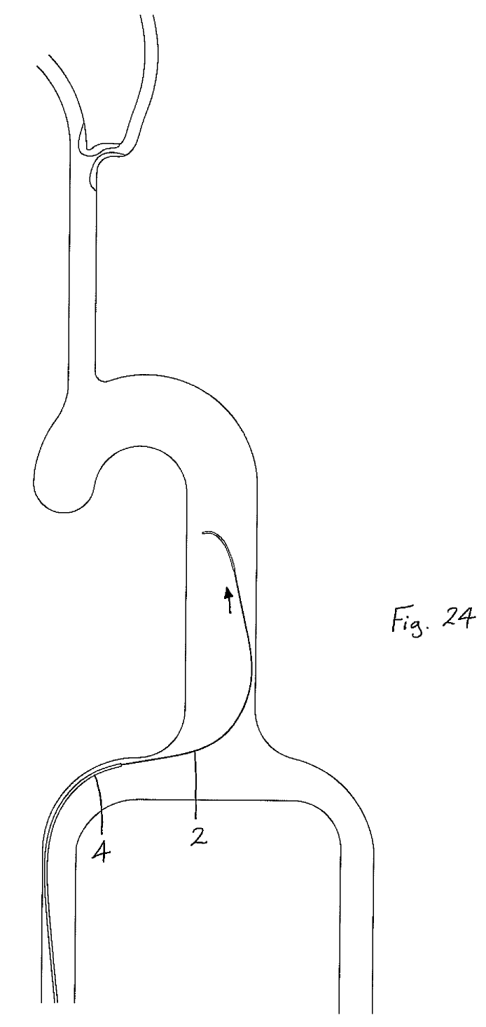

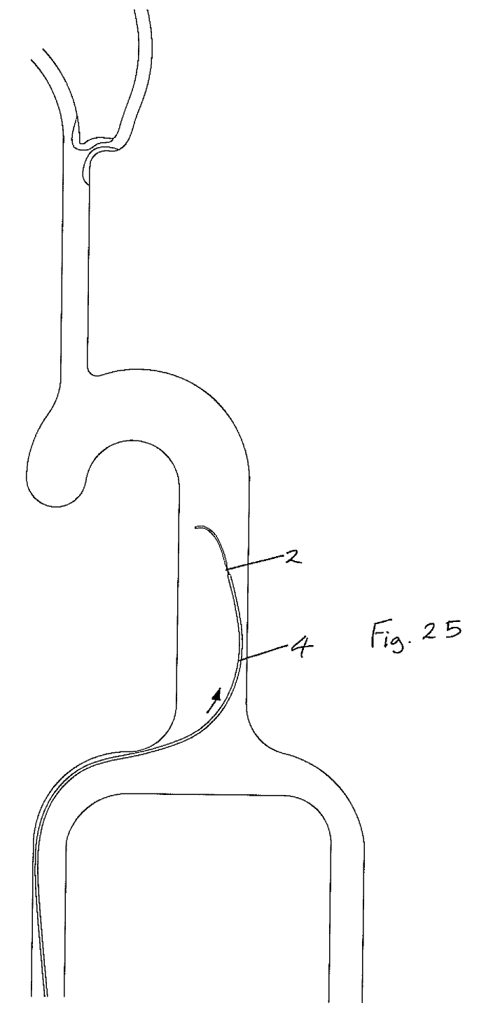

In this case the guide catheter 5 is not used. The guidewire 2, the delivery catheter 4 and the collapsed filter 1 are advanced together through the vasculature (FIG. 22). At a first bend in the vasculature, the guidewire 2 is advanced distally while the delivery catheter 4 and the collapsed filter 1 remain substantially stationary (FIGS. 23 and 24). The delivery catheter 4 and the collapsed filter 1 are then advanced distally over the guidewire 2 while the guidewire 2 remains substantially stationary (FIG. 25).

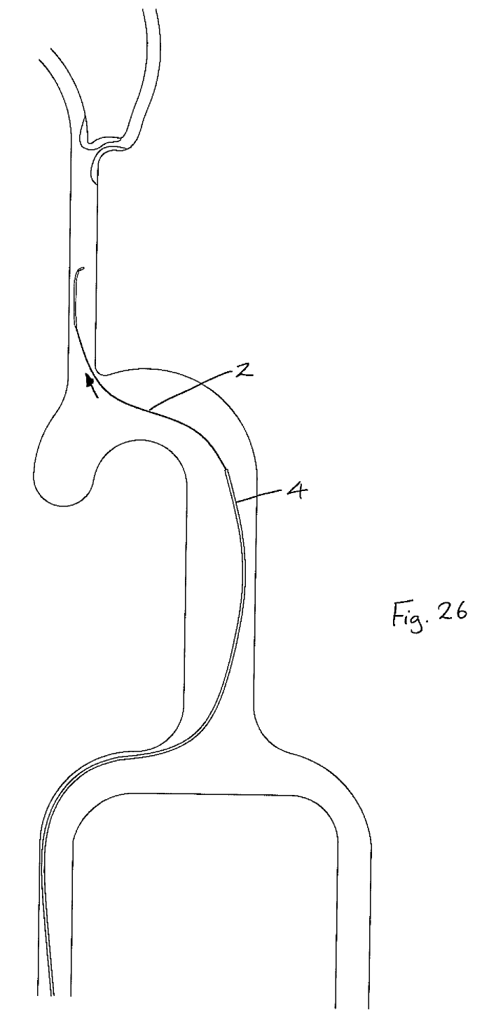

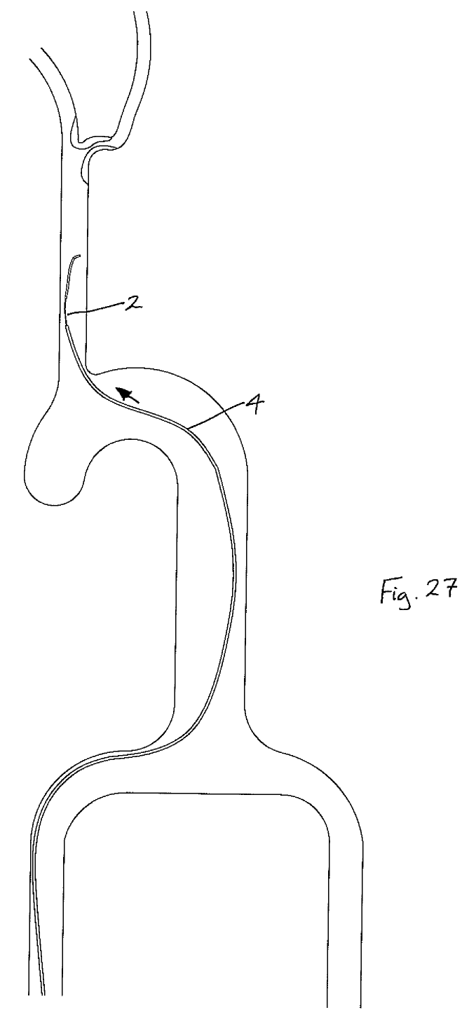

The guidewire 2, the delivery catheter 4 and the collapsed filter 1 are then advanced together further through the vasculature. At a second bend in the vasculature, the guidewire 2 is advanced distally while the delivery catheter 4 and the collapsed filter 1 remain substantially stationary (FIG. 26). The delivery catheter 4 and the collapsed filter 1 are then advanced distally over the guidewire 2 while the guidewire 2 remains substantially stationary (FIG. 27).

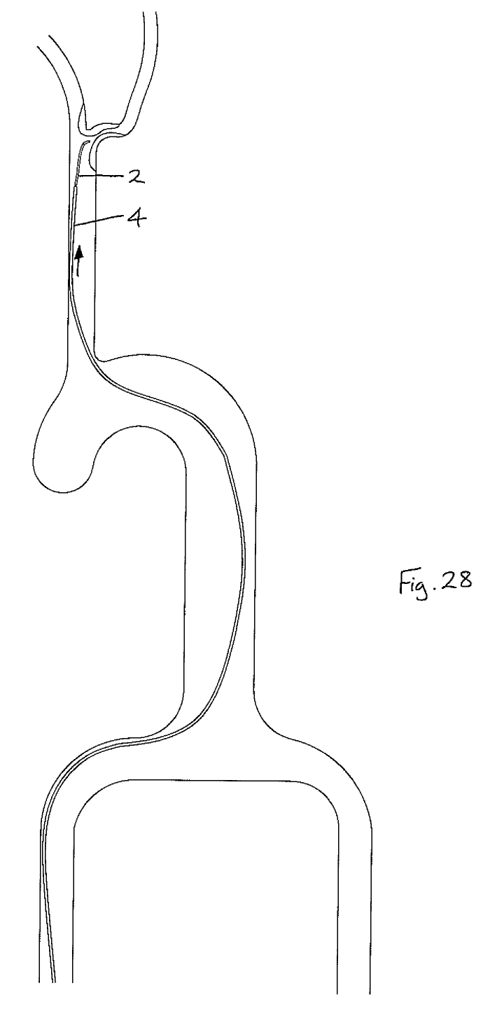

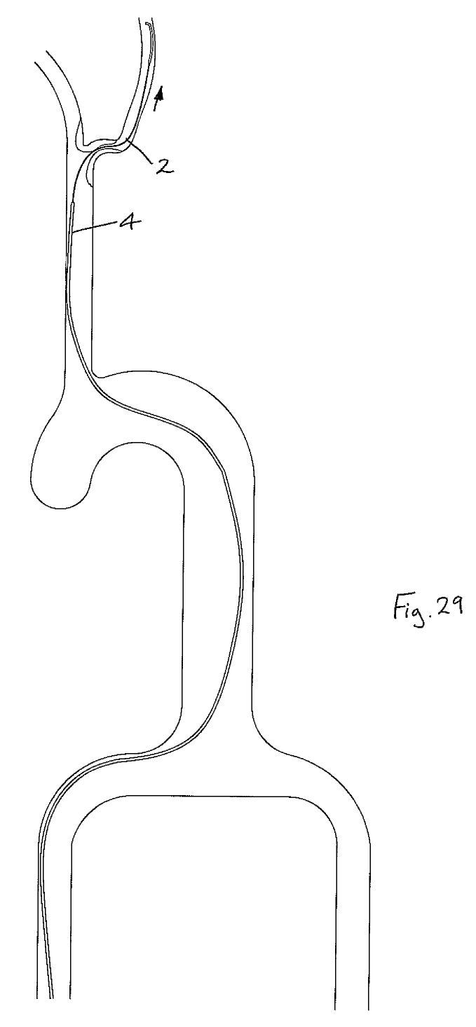

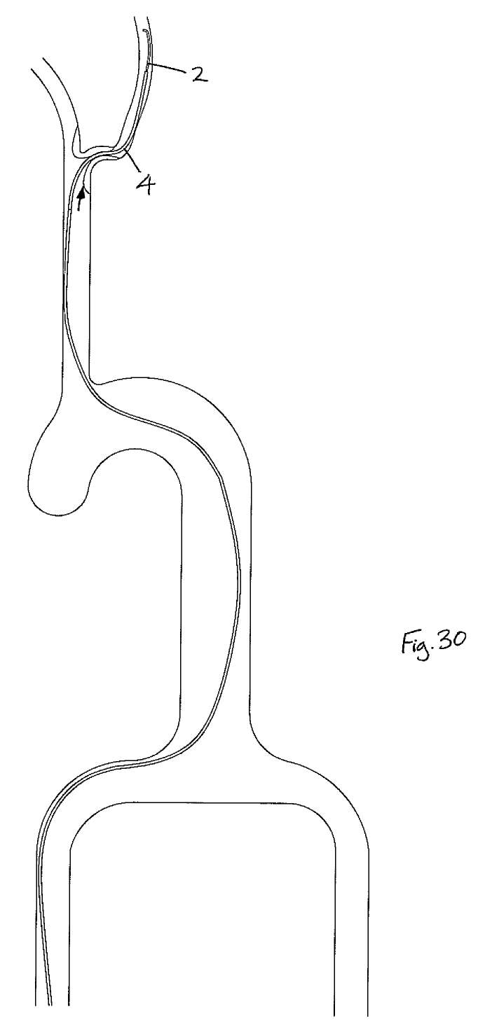

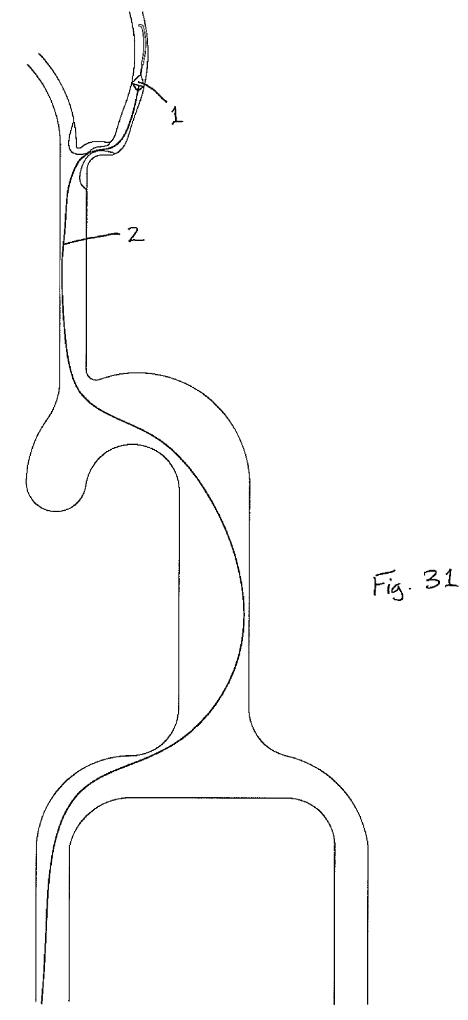

The delivery catheter 4, the guidewire 2 and the collapsed filer 1 are advanced together further through the vasculature to a point immediately proximally of the lesion (FIG. 28). The guidewire 2 is then advanced across the lesion (FIG. 29). During this advancement of the guidewire 2, the delivery catheter 4 and the collapsed filter 1 remain substantially stationary. When the guidewire 2 has crossed the lesion, the delivery catheter 4 and the collapsed filter 1 are then advanced across the lesion until the collapsed filer 1 is distal of the lesion (FIG. 30). The delivery catheter 4 is then withdrawn relative to the filter 1 to facilitate deployment of the filer 1 at the location distal of the lesion to ensure that any embolic material released during performance of an interventional procedure at the lesion will be captured and safely retained within the filter 1 (FIG. 31).

After an interventional or diagnostic procedure has been performed, the delivery catheter 4 is advanced again across the lesion or the area where the lesion was located and placed adjacent to the expanded filter 1. The guidewire 2 is then moved relative to the delivery catheter 4, wherein the feature 30 disposed on the guidewire 2 contacts the filter 1 therein imparting motion of the guidewire 2 to the filter 1. The filter 1 is then drawn into the distal end of the delivery catheter 4 through the motion of the guidewire 2. It is further contemplated that the distal end of the delivery catheter 4 may be configured to have an expandable portion which is configured to expand radially to capture the filter 1 and any materials which have been captured by the filter.

The invention is not limited to the embodiments hereinbefore described, with reference to the accompanying drawings, which may be varied in construction and detail.

1. A method for filtering embolic material from blood flowing in a vasculature during an interventional procedure, the method comprising the steps of:
providing an embolic protection filter having at least one proximal inlet and a plurality of distal outlets which are sized to capture embolic material while allowing blood to flow, the filter having a collapsed delivery configuration and an expanded configuration;
providing a guidewire, the filter being moveable relative to the guidewire;
advancing the filter in the collapsed delivery configuration through a vasculature to an intermediate location proximal of a desired treatment location;
advancing the guidewire through the vasculature from the intermediate location;
crossing the desired treatment location with the guidewire;
advancing the filter in the collapsed delivery configuration over the guidewire; and
deploying the filter distal to the treatment location.

2. A method as claimed in claim 1 comprising:
providing a delivery catheter for containing the filter in the collapsed configuration; and
advancing the delivery catheter, the filter and the guidewire to the intermediate location.

3-7. (canceled)

8. A method as claimed in claim 1 comprising the steps of:
advancing the guidewire from a first intermediate location to a further intermediate location which is proximal of the treatment location and, subsequently, advancing the filter in the collapsed delivery configuration over the guidewire towards the further intermediate location.

9. (canceled)

10. A method as claimed in claim 1 wherein the treatment location is in the carotid artery.

11. A method as claimed in claim 10 wherein the treatment location is at or adjacent to the carotid bifurcation.

12. A method as claimed in claim 10 wherein an intermediate location is at or adjacent to the aortic arch.

13. A method as claimed in claim 10 wherein the intermediate location is at or adjacent to the carotid take-off.

14. A method as claimed in claim 2 comprising:
providing the filter in the expanded configuration; and
loading the filter into the delivery catheter so that the delivery catheter contains the filter in the collapsed configuration.

15. A method as claimed in claim 14 comprising providing a guidewire extending through the filter in the delivery catheter.

16. A method as claimed in claim 15 wherein the filter and the delivery catheter are advanced along the guidewire.

17. A method as claimed in claim 16 further comprising the step of removing the delivery catheter from the filter at the deployment location.

18. A method as claimed in claim 17 wherein stored energy in the filter expands the filter on removal of the delivery catheter from the filter.

19. A method as claimed in claim 17 comprising withdrawing the delivery catheter from the deployment location.

20. A method as claimed in claim 19 wherein the delivery catheter is withdrawn from the deployment location after the deployment of the filter.

21. A method as claimed in claim 1 comprising introducing an interventional catheter over the guidewire to the treatment location for carrying out an interventional procedure, embolic material generated during the treatment procedure being captured by the deployed filter.

22. A method as claimed in claim 1 wherein the treatment location is a region of stenosis.

23. A method as claimed in claim 1 wherein the interventional procedure includes a balloon dilation of the stenosis while the filter is deployed.

24. A method as claimed in claim 1 wherein the interventional procedure includes placing a stent at the treatment location while the filter is deployed.

25. A method as claimed in claim 21 further comprising the steps of:
removing the interventional catheter from the treatment location; and
advancing a capture sheath over the guidewire.

26. A method as claimed in claim 25 further comprising the steps of:
engaging the filter with the capture sheath; and
withdrawing the filter and the capture sheath from the treatment location.

27. A method as claimed in claim 1 which further comprises the step of withdrawing the filter from the treatment location.

28. (canceled)

29. A method as claimed in claim 1 wherein the filter is slidably disposed on the guidewire when the filter is in an expanded deployed configuration.

30. A method as claimed in claim 1 wherein the filter is rotatably disposed on the guidewire when the filter is in an expanded deployed configuration.

31-33. (canceled)

34. A method as claimed in claim 1 wherein at least one stop is disposed on the guidewire.

35. A method as claimed in claim 34 wherein the at least one stop is a distal stop disposed on the guidewire distally of the filter.

36-52. (canceled)
